(19) United States
(12) Patent Application Publication
(10) Pub. No.: US 2010/0234590 A1
(43) Pub. Date: Sep. 16, 2010

(54) PROCESS FOR THE PURIFICATION NE OF OLANZAPINE

(76) Inventors: Abhay Gaitonde, Maharashtra (IN); Bindu Manojkumar, Maharashtra (IN); Rahul Bhalerao, Maharashtra (IN); Dattatraya Shinde, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/600,009

(22) PCT Filed: May 14, 2008
(86) PCT No.: PCT/GB08/50350

§ 371 Date: Jun. 1, 2010

(30) Foreign Application Priority Data
May 15, 2007 (IN) 918/MUM/2007

(51) Int. Cl. C07D 243/10; H01J 49/26; G01N 30/00

(52) U.S. Cl. 540/557; 250/282; 73/61.52

(57) ABSTRACT

The present invention relates to a novel process for the preparation of pharmaceutically pure olanzapine. The invention is also related to impurities obtained during the preparation of pharmaceutically pure olanzapine and methods for the detection of the impurities.

### CROSS-REFERENCE TO RELATED APPLICATION(s)

This application is a Section 371 National Stage Application of International No. PCT/GB2008/050350, filed 14 May 2008 and published as WO 2008/1391228 A3 on 20 Nov. 2008, which claims priority from the IN Patent Application No. 918/MUM/2007, filed 15 May 2007, the contents of which are incorporated herein in their entirety for all purposes.

### FIELD OF THE INVENTION

The present invention relates to a novel process for the preparation of pharmaceutically pure olanzapine. The invention is also related to impurities obtained during the preparation of pharmaceutically pure olanzapine and methods for the detection of the impurities.

### BACKGROUND OF THE INVENTION

Olanzapine is useful for treating psychotic patients and mild anxiety states. Olanzapine is chemically named 2-methyl-4-(4-methyl-1-piperazinyl)-10H-thieno[2,3-b][1.5]benzo-diazepine and has the following chemical structure:

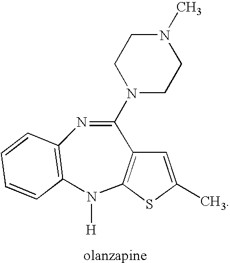

U.S. Pat. No. 5,229,382 discloses the preparation of olanzapine and pharmaceutically acceptable acid addition salts thereof, which have pharmaceutical properties particularly suitable in the treatment of disorders of the central nervous system. In particular, it discloses that the product prepared by the process disclosed therein is purified by chromatography on Florisil®, eluted with ethyl acetate and finally crystallized from acetonitrile. Such a purification technique is not useful for large scale manufacture.

The present inventors have found that when the process described in U.S. Pat. No. 5,229,382 is followed, olanzapine form I is not obtained. The above process consistently leads to olanzapine form II, even when olanzapine form I is used as a seeding agent. It has also been found that the product prepared by this process leads to a product with a pharmaceutically undesirable colouration.

WO 02/18390 describes a preparation of hydrates of olanzapine and their conversion into crystalline forms of olanzapine. The publication discloses a process for preparing olanzapine form I, which process comprises refluxing olanzapine with dichloromethane, decolourisation of the solution with carbon and isolation of the product by cooling and filtration. There is no mention of the amount of time that the olanzapine solution is treated with carbon, nor is there any mention of any impurities that may be present in the solution.

EP 0,733,634 relates to processes for preparing olanzapine and to lower alcohol solvates used in such methods. In particular, methanol, ethanol and isopropanol solvates are disclosed. The subject of this disclosure is as stated, the preparation of purer technical grade olanzapine, which process provides greater yields and fewer tedious separation steps. However, there is no mention of utilizing carbon as a purification technique, nor any mention of any impurities that may be present. EP 0,733,635, granted to the same applicants, relates to a polymorphic form of olanzapine designated form II. The problem of undesirable colouration is acknowledged in this disclosure where it is stated that “even carbon treatment of the olanzapine prepared using the methods described in the '382 patent does not remove all of the undesired colour”. Further, it is stated that the polymorphic form of the invention will contain less than 0.5% related substances. The International Committee for Harmonisation states that impurity levels for unknown related substances should be less than 0.1% before the expensive and onerous task of toxicological characterization. Thus, it will be apparent to the skilled person that the removal of impurities on a large scale constitutes a serious technical problem.

U.S. Pat. No. 6,906,062 also identifies the problem of an undesirably coloured olanzapine, but teaches away from using carbon purification methods by stating that prior art methods were not successful in removing undesired colour from olanzapine. U.S. Pat. No. 6,906,062 discloses olanzapine having stable colour on storage at ambient temperature. Also disclosed is a process for preparing olanzapine form I comprising two crystallizations, wherein at least one crystallization step comprises purification of the solution by treating with a solid adsorbent material wherein the solid adsorbent material is selected from alumina, silica, fullers earth and activated charcoal, and wherein the last crystallization step comprises subjecting the crystalline material to drying. The activated charcoal is added during the cooling stage of one of the two crystallization steps. This necessitates the need for filtering of the solution, an additional step which adds to the complexity of the disclosed process. Further, there is no mention of the types of impurities that are likely to be present.

WO 2004/056833 discloses a process for preparation of olanzapine form I, which comprises crystallization of olanzapine from a solution in dichloromethane, wherein before crystallization the solution is treated with silica gel at reflux temperature. Also disclosed is olanzapine form I substantially free of [1-(chloromethyl)-1-methyl-4-(2-methyl-10H-thieno[2.3b][1.5] benzodiazepin-4-yl]piperazin-1-ium chloride (impurity S) as well as a process for removal of impurity S. WO 2004/056833 further mentions that the process claimed in WO 02/18390 can lead to formation of additional impurities if the duration of the process of crystallization is extended.

WO 2005/090359 again highlights that treatment of carbon does not rid olanzapine products as prepared by the process disclosed in U.S. Pat. No. 5,229,382 of the undesirable colouration. WO 2005/090359 further states that olanzapine cannot be efficiently separated from its highly related impurities using repeated crystallization of crude olanzapine.

### SUMMARY OF THE INVENTION

As disclosed in the prior art described above, carbon treatment is utilized in order to remove or decrease the pharmaceutically unpalatable colouration of olanzapine and further as a means to remove impurities (related substances) associated with olanzapine during its preparation. The present inventors have observed that treating olanzapine with carbon as described in the prior art does obtain the desired colour improvement, but also generates impurities which, as have been clearly demonstrated in the prior art documents, are often difficult to remove by standard techniques known to the skilled person, such as recrystallization, precipitation and even carbon slurrying.

Accordingly there is provided a method of removing impurities from olanzapine utilising carbon, whilst maintaining the decolourisation effect of carbon.

In particular, the present inventors have identified the structure of an impurity generated during the treatment of olanzapine with carbon, namely impurity (I). Impurity (I), a dimer, was identified using Liquid Chromatography Mass Spectrometry (LCMS). According to one aspect of the invention, there is provided a compound having the formula (I):

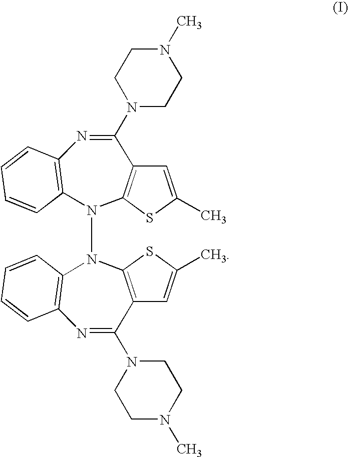

In an attempt to avoid the formation of this impurity during contact with carbon, the present inventors tried carbon treatment at room temperature as opposed to reflux temperatures. However, this treatment also resulted in formation of the dimer (I).

The present inventors have also found that treating a hot solution of olanzapine with carbon as disclosed in WO 02/18390 and U.S. Pat. No. 6,906,062 provides neither the removal of the undesirable impurity nor a lowering of its level. On the contrary, it was noticed that stirring the solution of olanzapine with carbon at room temperature or reflux temperature lead to generation of the dimer impurity. Hence, there is a need for developing an improved purification process for olanzapine, which does not generate any impurity, but is still effective in improving the colour of the final olanzapine product.

The difficulties encountered in the prior art for the preparation of pharmaceutically pure olanzapine have been successfully overcome by the present invention.

Accordingly, there is provided a process for the preparation of olanzapine, comprising the steps of:

(a) dissolving olanzapine or a salt thereof in a solvent;
(b) contacting the solution with carbon; and
(e) isolating olanzapine; characterized in that the contact of the solution with the carbon in step (b) is for a duration of about 15 minutes or less.

Preferably the process is for the preparation of pharmaceutically pure olanzapine. For the purposes of the present invention, the term “pharmaceutically pure” means that the olanzapine comprises less than 5% of any impurities, preferably less than 3%, more preferably less than 1%, more preferably less than 0.1%, more preferably less than 0.05%, more preferably less than 0.01% (as measured by HPLC). Preferably the olanzapine also comprises less than 5% of any olanzapine hydrates or solvates, preferably less than 3%, more preferably less than 1%, more preferably less than 0.1%, more preferably less than 0.05%, more preferably less than 0.01% (as measured by XRPD).

The present invention preferably employs activated charcoal as the carbon source to achieve colour improvement as well as the desired chemical purity of olanzapine. This has been achieved by exploiting the discovery that the prior art method of employing carbon resulted in increased levels of related impurities, and drastically reducing the time the olanzapine solution is in contact with the carbon does in fact reduce the levels of related impurities. Without wishing to be bound by theory, it is believed that carbon may be acting as a catalyst driving the formation of the related impurities rather than as an adsorbent removing said impurities from the solution. Preferably, the contact time is between about 3-10 minutes, more preferably between about 3-7 minutes, most preferably between 3-4 minutes.

It was further observed that increasing the duration of contact with the carbon actually results in an increase in impurity levels. Experiments performed by the inventors show that less stirring time than the conventional prior art contact time with the carbon resulted in a product with better purity. Thus, an advantage of the present invention is the removal of the dimer impurity as well as colour improvement by contacting a solution of olanzapine with carbon with very little contact time.

In a preferred embodiment, the activated charcoal is in a bed configuration. A particularly preferred embodiment of the charcoal bed is an activated charcoal cartridge. In such configurations, the solution comprising the olanzapine is allowed to pass through the charcoal bed. Embodiments comprising the charcoal bed configuration have the further advantage that the solution does not need to be filtered to remove the activated charcoal. An added advantage of this approach is its suitability to scale up to hundreds of kilos by using commercially available activated charcoal cartridges. In further embodiments, there is provided a process for the preparation of olanzapine according to the invention, wherein the charcoal bed is a commercially available charcoal bed. In a preferred embodiment, the olanzapine solution is passed through the charcoal bed at reduced pressure; in a particularly preferred embodiment the pressure required for vacuum filtration is 0.5-1 kg/cm², particularly 0.7-0.8 kg/cm².

In a preferred embodiment of the process according to the invention, in step (a), olanzapine is used. If an olanzapine salt is used in step (a), then olanzapine may be obtained by adjusting the pH of the solution.

In a preferred embodiment of the process according to the invention, in step (a), the olanzapine or a salt thereof is dissolved in a solvent at reflux temperature.

In a further preferred embodiment of the process according to the invention, the solvent used in step (a) is dichloromethane to prepare olanzapine form I, or alternatively the solvent used in step (a) is acetonitrile to prepare olanzapine form II. For the purposes of the present invention, olanzapine form I has an X-ray diffraction pattern comprising at least five peaks (preferably at least six, seven, eight, nine, ten, twelve, fifteen, twenty or more peaks) selected from peaks with d-values of about 9.94, 8.55, 8.24, 6.88, 6.37, 6.24, 5.58, 5.30, 4.98, 4.83, 4.72, 4.62, 4.53, 4.46, 4.29, 4.23, 4.08, 3.82, 3.74, 3.69, 3.58, 3.50, 3.33, 3.28, 3.21, 3.11, 3.05, 2.94, 2.81, 2.75, 2.65, 2.63 and 2.59±0.02, when copper Kα radiation is used. For the purposes of the present invention, olanzapine form II has an X-ray diffraction pattern comprising at least five peaks (preferably at least six, seven, eight, nine, ten, twelve, fifteen, twenty or more peaks) selected from peaks with d-values of about 10.26, 8.57, 7.47, 7.12, 6.14, 6.07, 5.48, 5.21, 5.12, 4.98, 4.76, 4.71, 4.47, 4.33, 4.22, 4.14, 3.98, 3.72, 3.56, 3.53, 3.38, 3.25, 3.12, 3.08, 3.06, 3.01, 2.87, 2.81, 2.72, 2.64 and 2.60±0.02, when copper Kα radiation is used.

In a preferred embodiment of the process according to the invention, there is provided a process wherein the olanzapine is isolated in step (c) by filtration and crystallization. Preferably the olanzapine is crystallized by cooling the filtrate. Preferably, the filtrate is cooled to between 0-5° C.

The process according to the invention is suitable for large scale manufacture, for example, for preparing olanzapine in batches of 1 kg, 10 kg, 50 kg, 100 kg, 200 kg, 500 kg, or more.

In another aspect according to the invention, there is provided olanzapine comprising less than 10% of a dimer having the formula (I):

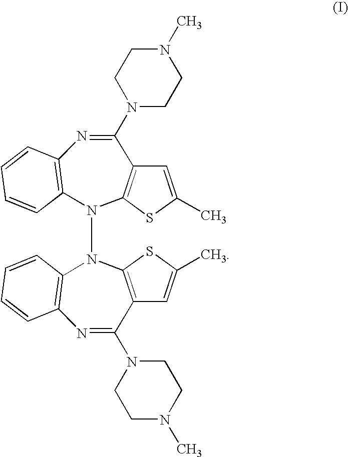

Preferably, the olanzapine comprises less than 5% of the dimer, more preferably less than 1%, more preferably less than 0.1%, more preferably less than 0.05%, more preferably less than 0.01%. Particularly preferred is olanzapine comprising undetectable amounts of the dimer (I).

Preferably the olanzapine is substantially pure, which means that the olanzapine comprises less than 5% of any impurities, preferably less than 3%, more preferably less than 1%, more preferably less than 0.1%, more preferably less than 0.05%, more preferably less than 0.01% (as measured by HPLC). Preferably the olanzapine also comprises less than 5% of any olanzapine hydrates or solvates, preferably less than 3%, more preferably less than 1%, more preferably less than 0.1%, more preferably less than 0.05%, more preferably less than 0.01% (as measured by XRPD).

In a further aspect, there is provided olanzapine form I comprising less than 0.1% related substances, preferably less than 0.05%, more preferably less than 0.01%. Preferably the olanzapine form I is substantially pure, which means that the olanzapine form I comprises less than 5% of any impurities, preferably less than 3%, more preferably less than 1%, more preferably less than 0.1%, more preferably less than 0.05%, more preferably less than 0.01% (as measured by HPLC), and that the olanzapine form I comprises less 10% of any other polymorphic forms, preferably less than 5%, more preferably less than 1%, more preferably less than 0.5%, more preferably less than 0.1% (as measured by XRPD). Preferably the olanzapine form I also comprises less 10% of any olanzapine hydrate or solvate forms, preferably less than 5%, more preferably less than 1%, more preferably less than 0.5%, more preferably less than 0.1% (as measured by XRPD).

In a further aspect, there is also provided olanzapine form II comprising less than 0.1% related substances, preferably less than 0.05%, more preferably less than 0.01%. Preferably the olanzapine form II is substantially pure, which means that the olanzapine form II comprises less than 5% of any impurities, preferably less than 3%, more preferably less than 1%, more preferably less than 0.1%, more preferably less than 0.05%, more preferably less than 0.01% (as measured by HPLC), and that the olanzapine form II comprises less 10% of any other polymorphic forms, preferably less than 5%, more preferably less than 1%, more preferably less than 0.5%, more preferably less than 0.1% (as measured by XRPD). Preferably the olanzapine form II also comprises less 10% of any olanzapine hydrate or solvate forms, preferably less than 5%, more preferably less than 1%, more preferably less than 0.5%, more preferably less than 0.1% (as measured by XRPD).

In a further aspect, there is provided a compound having the formula (I). In yet a further aspect, there is provided a method of detecting a compound having the formula (I), comprising using Liquid Chromatography Mass Spectrometry.

### DETAILED DESCRIPTION OF THE INVENTION

The present invention relates to the discovery that the presence of impurities is affected by the length of time an olanzapine solution is in contact with carbon. Decolourisation is desired as olanzapine per se is a deep brown colour that is pharmaceutically unpalatable. Although the colouration may not be dangerous, end users are put off with a deleterious effect on patient compliance. Many solutions have been put forward including coating the final dosage formulation of the olanzapine particles. However coatings suitable to hide the brown colouration often contain iron-based pigments and there is an industry-wide consensus to reduce the intake of such pigments.

Another solution is to remove or reduce the colouration during manufacture of olanzapine. In this case, charcoal is usually added to the olanzapine solution to form a slurry. It has now been found by the present inventors that increased contact time with charcoal will result in an increased impurity profile, particularly of the dimer impurity (I).

Accordingly, there is provided a process, wherein the contact time of the olanzapine solution with the charcoal is regulated, such that a minimal amount of impurity is formed. In a particularly preferred aspect, the minimal contact time is effected by the use of a charcoal bed. The bed is such that the olanzapine solution can pass through the bed within a predetermined length of time. Of course, it is understood that there is a compromise between the length of contact time and the amount of time needed to reduce the colouration of the olanzapine solution. In this regard, the inventors have found that the solution should be in contact with the charcoal for no longer than 15 minutes, preferably between 3-10 minutes, more preferably between 3-7 minutes, and most preferred between 3-4 minutes. The charcoal bed in preferred embodiments may be of any configuration allowing the olanzapine solution to flow through. The configuration or density can be altered within the scope of the appended claims to achieve such a result. Indeed, a number of commercial charcoal beds may be suitably employed in the working of the invention.

The starting olanzapine solution can be prepared by any of the methods known in the art including those described in U.S. Pat. Nos. 5,736,541; 5,229,382; 6,348,458; WO 04/58773, and US Patent Publication No. 2002/0086993. Further, olanzapine starting material can be of any form or purity, including crude or technical grade olanzapine, which in certain embodiments are preferably form I or form II, or other hydrated or solvated forms of olanzapine.

Preferably, the solution of olanzapine may be obtained by dissolving olanzapine in a suitable solvent for preparing form I or form II. In particularly preferred embodiments to prepare olanzapine form I, the solvent used is dichloromethane. In alternative embodiments, acetonitrile is used to prepare olanzapine form II. Alternatively, the solution of olanzapine may be obtained directly from a reaction in which olanzapine is formed. If a suspension is obtained in a solvent, the suspension containing olanzapine may be heated to obtain a solution.

Once the solution has been through the carbon treatment, for example, through the charcoal bed in certain preferred embodiments, or minimal contact in a charcoal slurry such that impurities are not formed in alternative embodiments, the resultant filtrate can be subjected to a number of techniques known in the art to isolate the pharmaceutically pure olanzapine depending on the solvent employed. In preferred embodiments, the pharmaceutically pure olanzapine can be isolated by filtration and crystallization. The filtrate may in certain embodiments be stirred from ambient temperature to about 0° C., preferably between 0-5° C. for a time sufficient to complete crystallization. The crystals may be removed from the solution by techniques including, for example, filtration, filtration under vacuum, decantation and centrifugation. The product may be washed and dried by conventional methods to obtain the desired olanzapine form.

The isolated olanzapine is generally pure or substantially free of other substances, impurities, hydrates or solvates, and is typically, though not necessarily, at least 97% pure, and usually at least 99% pure (as measured by HPLC or XRPD). The isolated olanzapine is also generally morphologically pure form I or form II depending on the solvent employed, for example, at least 90% olanzapine form I or II, preferably at least 95% form I or II, more preferably at least 99% form I or II, and most preferably essentially 100% form I or II, based on the total weight of crystalline olanzapine. The olanzapine form I or II produced by the present invention preferably shows no indication of the other olanzapine form, and more preferably no indication of any other olanzapine form, by X-ray powder diffraction analysis.

Of course, it will be understood that as well as the crystalline forms described above, the teaching of the invention can be applied to other forms, such as amorphous or liquid crystal or any form of olanzapine, the only limitation being the ability to form a solution in a solvent.

The pure olanzapine of the present invention may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, intravenous, and ophthalmic) administration. The oral dosage forms may include solid dosage forms, like powders, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

The olanzapine can be administered for the treatment of schizophrenia, or acute mixed or manic episodes associated with bipolar I disorder.

The following paragraphs enumerated consecutively from 1 through 28 provide for various aspects of the present invention. In one embodiment, the present invention provides:

1. A process for the preparation of olanzapine, comprising the steps of:
(a) dissolving olanzapine or a salt thereof in a solvent;
(b) contacting the solution with carbon; and
(c) isolating olanzapine; characterized in that the contact of the solution with the carbon in step (b) is for a duration of about 15 minutes or less.
2. A process according to paragraph 1, wherein the process is for the preparation of pharmaceutically pure olanzapine.
3. A process according to paragraph 1 or 2, wherein in step (a) olanzapine or a salt thereof is dissolved in a solvent at reflux temperature.
4. A process according to any one of the preceding paragraphs, wherein in step (a) olanzapine is used.
5. A process according to any one of the preceding paragraphs, wherein the carbon is in the form of activated charcoal.
6. A process according to any one of the preceding paragraphs, wherein the carbon is in the form of a charcoal bed.
7. A process according to paragraph 6, wherein the carbon is in the form of an activated charcoal bed.
8. A process according to any one of the preceding paragraphs, wherein the carbon is in the form of an activated charcoal cartridge.
9. A process according to any one of the preceding paragraphs, wherein the contact time of the carbon with the solution is between about 3-10 minutes.
10. A process according to any one of the preceding paragraphs, wherein the contact time of the carbon with the solution is between about 3-7 minutes.
11. A process according to any one of the preceding paragraphs, wherein the contact time of the carbon with the solution is between about 3-4 minutes.
12. A process according to any one of paragraphs 1 to 11, wherein the solvent used in step (a) is dichloromethane and the olanzapine prepared is olanzapine form I.
13. A process according to paragraph 12, wherein the olanzapine form I has an X-ray diffraction pattern comprising at least five peaks selected from peaks with d-values of about 9.94, 8.55, 8.24, 6.88, 6.37, 6.24, 5.58, 5.30, 4.98, 4.83, 4.72, 4.62, 4.53, 4.46, 4.29, 4.23, 4.08, 3.82, 3.74, 3.69, 3.58, 3.50, 3.33, 3.28, 3.21, 3.11, 3.05, 2.94, 2.81, 2.75, 2.65, 2.63 and 2.59±0.02, when copper Kα radiation is used.
14. A process according to any one of paragraphs 1 to 11, wherein the solvent used in step (a) is acetonitrile and the olanzapine prepared is olanzapine form 11.
15. A process according to paragraph 14, wherein the olanzapine form II has an X-ray diffraction pattern comprising at least five peaks selected from peaks with d-values of about 10.26, 8.57, 7.47, 7.12, 6.14, 6.07, 5.48, 5.21, 5.12, 4.98, 4.76, 4.71, 4.47, 4.33, 4.22, 4.14, 3.98, 3.72, 3.56, 3.53, 3.38, 3.25, 3.12, 3.08, 3.06, 3.01, 2.87, 2.81, 2.72, 2.64 and 2.60±0.02, when copper Kα radiation is used.
16. A process according to any one of the preceding paragraphs, wherein the olanzapine is isolated in step (c) by filtration and crystallization.
17. A process according to paragraph 16, wherein the olanzapine is crystallized by cooling the filtrate.
18. A process according to paragraph 17, wherein the filtrate is cooled to between 0-5° C.
19. Olanzapine comprising less than 10% of a dimer having the formula (I):

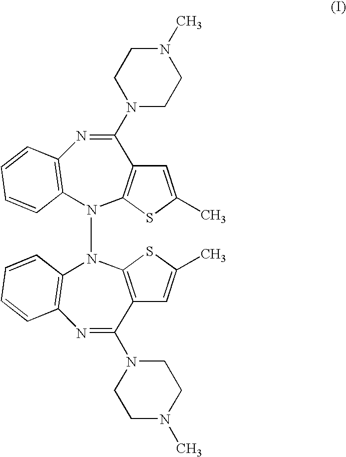

20. Olanzapine according to paragraph 19, comprising less than 5% of the dimer having the formula (I).
21. Olanzapine according to paragraph 20, comprising less than 1% of the dimer having the formula (I).
22. Olanzapine according to paragraph 21, comprising less than 0.1% of the dimer having the formula (I).
23. Olanzapine according to paragraph 22, comprising less than 0.05% of the dimer having the formula (I).
24. Olanzapine according to paragraph 23, comprising less than 0.01% of the dimer having the formula (I).
25. Olanzapine form I comprising less than 0.1% related substances.
26. Olanzapine form II comprising less than 0.1% related substances.
27. A compound having the formula (I):

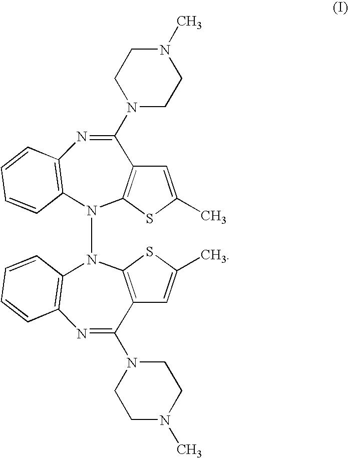

28. A method of detecting a compound according to paragraph 27, comprising using Liquid Chromatography Mass Spectrometry.

### EXAMPLES

### Comparative Example

The impurity profile of crude olanzapine form II obtained by the process described in U.S. Pat. No. 5,229,382 is as given in Table 1 below.

TABLE 1

| RRT | 0.17 | 0.46 | 0.78 | 1 | 1.27 | 1.37 | 2.11 |
|---|---|---|---|---|---|---|---|
| % Area | 0.33 | 0.03 | 0.09 | 98.89 | 0.05 | 0.06 | 0.53 |

### Example 1

### Purification of Polymorphic Form I from Crude Olanzapine

100 g (1 eq) of crude olanzapine form I was dissolved in 500 ml (5 vol) of dichloromethane at reflux temperature. The hot solution was passed through a charcoal bed (2.5% w/w) under reduced pressure. The filtrate obtained was cooled to 0-5° C. with stirring to obtain olanzapine form I as bright yellow coloured solid. The slurry was filtered.

### Yield=80%

### Purity=99.89%

The dimer impurity (I) was not detectable using HPLC.

### Example 2

### Purification of Polymorphic Form II from Crude Olanzapine

100 g (1 eq) of crude olanzapine form H was dissolved in 1200 m1 (12 vol) of acetonitrile at reflux temperature. The hot solution was passed through a charcoal bed (2.5% w/w) under reduced pressure. The filtrate obtained was cooled to 0-5° C. with stirring to obtain olanzapine form II as bright yellow coloured solid. The slurry was filtered.

### Yield=83.53%

### Purity=99.95%

The dimer impurity (I) was not detectable using HPLC.

The impurity profile of the olanzapine form II obtained in example 2 is as given in Table 2 below.

TABLE 2

| | RRT | | |
|---|---|---|---|
| | 0.46 | 0.78 | 1 |
| % Area | 0.02 | 0.03 | 99.95 |

It can be seen from the results of examples 1 and 2 that processes according to the invention not only completely remove the dimer impurity (I), but further cause a decrease in the total level of impurities formed as a result of the prior art processes for preparing olanzapine.

While the present invention has been described in terms of its specific embodiments, certain modifications and equivalents will be apparent to those skilled in the art and are intended to be included within the scope of the present invention.

What is claimed is:

1. A process for the preparation of olanzapine, comprising the steps of:
(a) dissolving olanzapine or a salt thereof in a solvent;
(b) contacting the solution with carbon; and
(c) isolating olanzapine;
characterized in that the contact of the solution with the carbon in step (b) is for a duration of about 15 minutes or less.

2. A process according to claim 1, wherein the process is for the preparation of pharmaceutically pure olanzapine.

3. A process according to claim 1, wherein in step (a) olanzapine or a salt thereof is dissolved in a solvent at reflux temperature.

4. A process according to claim 1, wherein in step (a) olanzapine is used.

5. A process according to claim 1, wherein the carbon is in the form of:
(i) activated charcoal; and/or
(ii) a charcoal bed; and/or
(iii) an activated charcoal bed; and/or
(iv) an activated charcoal cartridge.

6. A process according to claim 1, wherein the contact time of the carbon with the solution is:
between about 3-10 minutes; and/or
(ii) between about 3-7 minutes; and/or
(iii) between about 3-4 minutes.

7. A process according to claim 1, wherein the solvent used in step (a) is dichloromethane and the olanzapine prepared is:
(i) olanzapine form I; and/or
(ii) olanzapine form I having an X-ray diffraction pattern comprising at least five peaks selected from peaks with d-values of about 9.94, 8.55, 8.24, 6.88, 6.37, 6.24, 5.58, 5.30, 4.98, 4.83, 4.72, 4.62, 4.53, 4.46, 4.29, 4.23, 4.08, 3.82, 3.74, 3.69, 3.58, 3.50, 3.33, 3.28, 3.21, 3.11, 3.05, 2.94, 2.81, 2.75, 2.65, 2.63 and 2.59±0.02, when copper Kα radiation is used.

8. A process according to claim 1, wherein the solvent used in step (a) is acetonitrile and the olanzapine prepared is:
(i) olanzapine form II; and/or
(ii) olanzapine form II having an X-ray diffraction pattern comprising at least five peaks selected from peaks with d-values of about 10.26, 8.57, 7.47, 7.12, 6.14, 6.07, 5.48, 5.21, 5.12, 4.98, 4.76, 4.71, 4.47, 4.33, 4.22, 4.14, 3.98, 3.72, 3.56, 3.53, 3.38, 3.25, 3.12, 3.08, 3.06, 3.01, 2.87, 2.81, 2.72, 2.64 and 2.60±0.02, when copper Kα radiation is used.

9. A process according to claim 1, wherein the olanzapine is isolated in step (c) by:
(i) filtration and crystallization; and/or
(ii) filtration and crystallization, wherein the olanzapine is crystallized by cooling the filtrate; and/or
(iii) filtration and crystallization, wherein the olanzapine is crystallized by cooling the filtrate to between 0-5° C.

10. Olanzapine comprising less than 10% of a dimer having the formula (I):

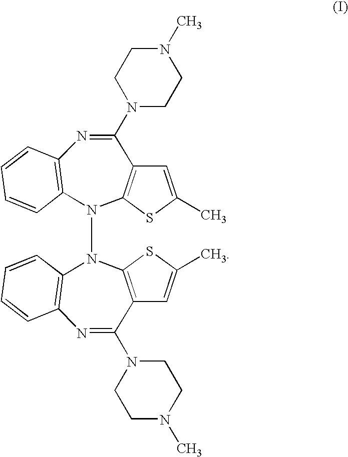

11. Olanzapine according to claim 10, comprising less than 5%, or less than 1%, or less than 0.1%, or less than 0.05%, or less than 0.01% of the dimer having the formula (I).

12. Olanzapine form I comprising less than 0.1% related substances.

13. Olanzapine form II comprising less than 0.1% related substances.

14. A compound having the formula (I):

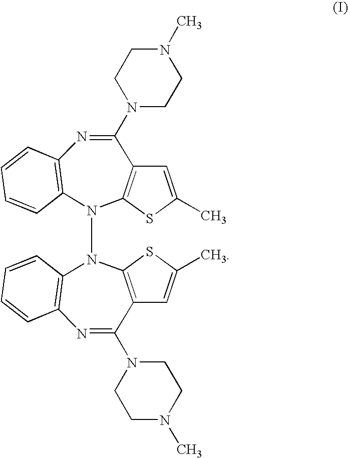

15. A method of detecting a compound according to claim 14, comprising using Liquid Chromatography Mass Spectrometry.
